(19) United States
(12) Patent Application Publication
(10) Pub. No.: US 2012/0296248 A1
(43) Pub. Date: Nov. 22, 2012

(54) SUPPORT BODY FOR KNEE ORTHOSES AND/OR KNEE JOINT BRACES

(75) Inventors: Kurt Wayd, Wien (AT); Josef Grafinger, Wien (AT)

(73) Assignee: KURT WAYD

(21) Appl. No.: 13/473,185

(22) Filed: May 16, 2012

(30) Foreign Application Priority Data
May 19, 2011 (AT) A 719/2011

(51) Int. Cl. A61F 5/00

(52) U.S. Cl. 602/16

(57) ABSTRACT

A support body (1) for knee orthoses and/or knee joint braces in the form of a splint (2) which extends along and partially encloses the lower leg of a wearer and is connected at the upper end to an orthosis hinge or a hinge of a knee joint brace and which is provided at a lower end with a foot part (3) receiving at least the heel of a wearer. At the upper end of the splint (2), two connection points (4) for the knee orthosis and/or knee joint braces are arranged laterally to the wearer's leg, which connection points transition into a section which runs downward at the wearer's shin bone along the leg and which, on its part, transitions again above the wearer's ankle region into two connecting parts (6) which run laterally to the leg to the foot part (3).

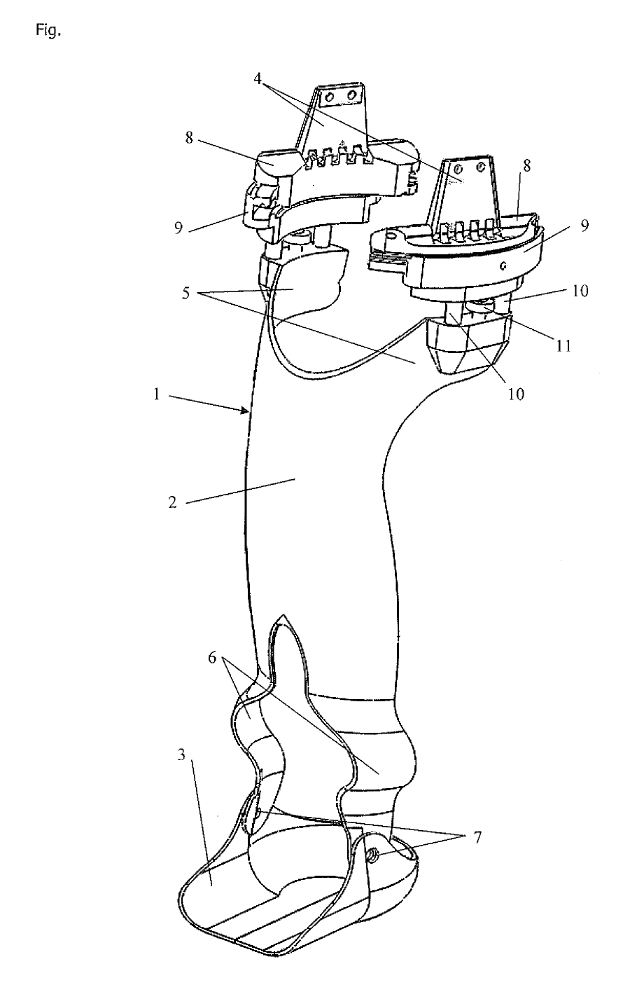

The invention relates to a support body for knee orthoses and/or knee joint braces in the form of a splint which extends along and partially encloses the lower leg and is connected at the upper end to an orthosis hinge or a hinge of the knee joint brace and which is provided at the lower end with a foot part receiving at least the heel.

Such constructions are known in a variety of embodiments. For example, CA 2543217 discloses a leg orthosis that offers a brace extending along the upper leg and the lower leg up to a foot part. The supporting structure can be fixed on the upper leg and the lower leg with straps which entirely enclose these leg sections which, however, results in different firmness of the fixation depending of the degree of movement of the muscle contraction in the leg.

An object of the present invention is a construction with which despite optimal supporting effect, the movability of the leg is impeded as little as possible.

In order to achieve this object, it is provided according to the invention that at the upper end of the splint, two connection points for the knee orthosis and/or knee joint braces are provided arranged laterally to the leg, which connection points transition into a section which runs downward at the shin bone along the leg and which, on its part, transitions again above the ankle region into two connecting parts which run laterally to the leg to the foot part.

According to an advantageous embodiment of the invention it is provided that the shin bone section is curved or bent around the leg so that the leg is covered over a portion of its circumference by said section.

Another embodiment is additionally characterized in that in the connecting parts below ankle level in each case one hinge for a pivotable connection between shin bone section and foot part is provided.

Advantageously, it is provided here that the axis of the hinges in the connecting parts is situated farther toward the heel on the outer side of the foot than on the inner side of the foot.

According to the invention, a support body can further be characterized in that the orthosis hinge or the hinge of the knee joint brace and the splint are connected to each other via two monocentric hinges, the axes of which are substantially oriented in the plane of the bending movement of the knee joint.

Yet another embodiment of the invention provides that the orthosis hinge or the knee joint brace and the splint are connected to each other via two hinge elements which allow a rotational movement between the support body and the orthosis hinge or knee brace hinge about the axis of the lower leg.

An advantageous variant is characterized in that each hinge element consists of two portions guided inside each other or against each along the path of a circle segment, wherein the circle segments of both hinge elements belong to a circle having its center in the central region of the leg.

Suitably, another embodiment of the invention is characterized in that a device for adjusting the gap between the orthosis hinge or knee joint brace hinge and the shin bone splint is provided at least at one of the connection points, preferably at both connection points.

Preferably, the monocentric hinges are arranged above the hinge elements for the rotational movement.

The one or each gap adjustment device can also be arranged below each provided hinge or hinge element.

In the following description, the invention shall be explained in more detail, also by means of a specific exemplary embodiment wherein, however, these illustrations do not represent any restrictions.

The FIGURE in the drawing shows a view of an embodiment of a support body according to the invention.

According to the invention, the support body 1 comprises a splint 2 which extends along and partially encloses the lower leg and which is connected at the upper end to an orthosis hinge or a knee joint brace hinge (not illustrated). A foot part 3 receiving at least the heel is movably secured at the lower end.

In order to optimally support the weight of the wearer during the use, two connection points 4 for the knee orthosis and/or knee joint braces are provided laterally to the leg at the upper end of the splint 2. From these connection points 4, two lateral branches 5 of the support body 1 run laterally downward along the leg and toward the front and transition into the splint's 2 section running downward along the leg, which section is advantageously formed curved or bent around the leg so that the leg is covered in the front over a portion of its circumference by the splint 2 wherein, however, the rear region of the leg remains uncovered. In the front, the splint 2 rests with its preferably slightly padded inner side directly against the shin bone. Preferably, this section is formed in one piece with the two lateral branches 5. In this manner, a lightweight construction with the required rigidity can be ensured.

Above the ankle region, the splint 2 also divides into two lateral connecting parts 6 which run laterally to the leg to the foot part 3. Advantageously, the connecting parts 6 are anatomically bulged outward corresponding to the ankle joint.

In the connecting parts 6, below the ankle level, in each case one hinge 7 is provided through which the foot part 3 is pivotably secured on the support body 1. Thus, the hinges 7 lie approximately 30 to 70 mm, preferably approximately 55 mm, above the sole of the foot part 3. Furthermore, it is provided that the axis of the hinges 7 are oriented in such a manner that it is shifted farther toward the heel on the outside of the foot than on the inside of the foot. From the rear end of the foot part 3 against which the heel rests, the axis of the hinges is shifted forward at the outer side of the foot by approximately 40 to 70 mm, preferably approximately 55 mm, and shifted forward at the inner side of the foot by a further 10 to 20 mm, preferably 15 mm.

The orthosis hinge or knee joint brace hinge and the lateral branches 5 can be connected to each other via two monocentric hinges 8, the axes of which are substantially oriented in the plane of the bending movement of the knee joint. Additionally or also alternatively to this, the orthosis hinge or knee joint brace hinge and the lateral branches 5 of the support body 1 can also be connected to each other via two hinge elements 9 which enable a rotational movement between the support body 1 and the orthosis hinge or knee joint brace hinge or the connection points 4 thereof about the axis of the lower leg. Each of these hinge elements 9 consists of two portions which can be moved with respect to each other, namely are guided inside each other or along a path of a circle segment, wherein the circle segments of both joint elements 9 belong to a circle having its center in the central region of the leg. Preferably, the monocentric hinges 8 are arranged above the hinge elements 9 for the rotational movements.

Additionally or also alternatively to the two above-mentioned hinges 8, 9, the support body 1 can be provided with a device for adjusting the gap between the orthosis hinge or knee joint brace hinge and the shin bone splint 2 at least at one of the two branches 5, preferably, of course, at both branches 5. An example for such a device is illustrated in the FIGURE of the drawing and comprises at each branch 5 two guide pins 10 which run between the end of the lateral branch 5 of the support body 1 and the structure arranged thereabove, here the hinge element 9, and guide the two parts 5, 9 linearly to each other. Adjusting the gap between the hinge element 9 and the lateral branch S is performed with an adjusting screw 11 which engages with oppositely running threads in the two parts 5, 9. The one or each device 10, 11 for gap adjustment can be arranged below each provided hinge 8 or hinge element 9.

1. A support body for knee orthoses and/or knee joint braces in the form of a splint (2) which extends along and partially encloses a lower leg and is connected at an upper end to an orthosis hinge or a hinge of a knee joint brace and which is provided at a lower end with a foot part (3) receiving at least a heel of a wearer, wherein at the upper end of the splint (2), two connection points (4) for the knee orthosis and/or knee joint braces are provided arranged laterally to the leg, which connection points transition into a shin bone section which runs downward at a shin bone along the leg of a wearer and which, on its part, transitions again above an ankle region into two connecting parts (6) which run laterally to the leg to the foot part (3).

2. The support body according to claim 1, wherein the shin bone section is curved or bent around the leg so that the leg is covered over a portion of its circumference by the section.

3. The support body according to claim 1, wherein in the connecting parts (6) below the ankle, in each case one hinge (7) is provided for a pivotable connection between the shin bone section and the foot part (3).

4. The support body according to claim 3, an axis of the joints (7) in the connecting parts (6) is situated farther toward the heel on an outer side of the foot than on an inner side of the foot.

5. The support body according to claim 1, wherein the orthosis hinge or knee joint brace hinge and the splint (2) are connected to each other via two monocentric hinges (8) having axes which are substantially oriented in a plane of bending movement of the knee joint.

6. The support body according to claim 1, wherein the orthosis hinge or knee joint brace hinge and the splint (2) are connected to each other via two hinge elements (9) which enable a rotational movement between the support body and the orthosis hinge or knee joint brace hinge about an axis of the lower leg.

7. The support body according to claim 6, wherein each hinge element (9) consists of two portions which are guided inside each other or against each other along a path of a circle segment, wherein the circle segments of both hinge elements belong to a circle having its center in a central region of the leg.

8. The support body according to claim 1, including a device (10, 11) for adjusting a gap between the orthosis hinge or knee joint brace hinge and the shin bone splint (2) at least at one of the connection points.

9. The support body according to claim 6, wherein monocentric hinges (8) are arranged above the hinge elements (9) for the rotational movement.

10. The support body according to claim 8, wherein the one or each device for adjusting the gap is arranged below each provided hinge (8, 9) or hinge element.
